(19) United States
(12) Patent Application Publication
(10) Pub. No.: US 2021/0290040 A1
(43) Pub. Date: Sep. 23, 2021

(54) Disposable Handle Assembly and Testing Device

(71) Applicant: TELEFLEX MEDICAL INCORPORATED, Morrisville, NC (US)

(72) Inventors: Aviram ELBAZ, Zoran (IL); Tamer ABU-AITA, Taybie (IL); Aaron ZICKEFOOSE, Fuquay-Varna, NC (US)

(21) Appl. No.: 16/321,624

(22) PCT Filed: Aug. 29, 2017
(86) PCT No.: PCT/US17/49104

§ 371 Date: Jan. 29, 2019

Related U.S. Application Data
(60) Provisional application No. 62/368,617, filed on Jul. 29, 2016.

(51) Int. Cl. A61B 1/00

(52) U.S. Cl. CPC A61B 1/00066; A61B 1/00032; A61B 1/267; A61B 1/00057; A61B 1/00055

(57) ABSTRACT

A disposable handle assembly may include a housing including an end wall at a distal end of the housing and first extension walls extending from the end wall, an end cap attached to a second extension wall on a proximal end of the housing, and a power source positioned within the housing between the distal end and the end cap. The disposable handle assembly may include a pin positioned within the housing, a circuit wire positioned within the housing, and a plate attachment positioned on the end wall between the first extension walls. The circuit wire may be in electrical contact with the contact plate and an end of the power source received by the end cap. The pin and the contact plate may be electrically connected outside of the housing and close an electrical circuit of the disposable handle assembly.

### CROSS-REFERENCE TO RELATED APPLICATION

This application claims priority to U.S. Provisional Patent Application No. 62/368,617 filed Jul. 29, 2016, the entire contents of which are incorporated herein by reference.

### TECHNICAL FIELD

The present disclosure relates generally to a disposable handle assembly and a testing device for testing the operational capability of the disposable handle assembly, which may be provided in an closed packaging that maintains the disposable handle assembly in a state that is safe and suitable for an intended use.

### BACKGROUND

Disposable (e.g. “single-use”) instruments and devices are often used in medical procedures. Inexpensive devices that can be maintained in packaging can be substituted for more expensive “multi-use” devices that must be sterilized prior to each use during a medical procedures. Sterilizing these multi-use devices and instruments can take time, be expensive, and may come with an increased risk of contamination if an error occurs during a sterilization process. As a result, disposable devices and instruments may be preferable for some procedures, especially those which are performed frequently.

Some such devices and instruments may include a power source which actuates some component of the device or instrument. For example, dual component laryngoscopes, such as those that are ISO 7376 compatible, may include a laryngoscope blade and a handle to which the laryngoscope blade can be snap fit to. Such dual component laryngoscopes may include a power source and a light source that emits light when connected to the power source. Many devices or instruments, including the dual component laryngoscopes discussed above, may be provided in closed packaging that must be permanently separated and discarded in order to use the device or instrument. In cases in which the device or instrument includes a power source for actuating a component, either of the device or instrument or of another device or instrument, the operative capacity of the device or instrument cannot be known or tested until it is removed from the closed packaging and put into use.

Disposable devices and instruments including a power source and/or a component actuated by a power source may be susceptible to having the power source or component used up, damaged, or otherwise rendered inoperable while in a closed packaging. For example, a device that includes a component that is moved into a position to be electrically connected to a power source may be moved into the position within a closed packing due to movement of the device or movement of other devices packaged with the device while the device is in transit. As such, the device may be inadvertently turned ON, and a power source such as a battery, may be completely depleted before a clinician or other operator opens the closed packaging and removes the device. In situations in which the device is about to be used in an actual procedure that is in process, the clinician or operator will spend time opening the closed packaging, removing the device from the previously closed packaging, and setting up the device to operate, only to realize when the setup was complete that the device was inoperable and that time had been wasted. Being able to determine the device was inoperable before being removed from the closed packaging would reduce the amount of time lost, which may beneficial in the course of performing a medical procedure.

In view of the foregoing, there exists a need for a device and a method that permits a device, such as a handle for a medical device or instrument, to be tested while being located in a closed packaging. These and other needs are addressed by the present disclosure.

### SUMMARY

According to an aspect of the present disclosure, a disposable handle assembly includes a housing including an end wall at a distal end of the housing and first extension walls extending from the end wall, and an end cap attached to a second extension wall on a proximal end of the housing. The disposable handle assembly further includes a power source positioned within the housing between the distal end and the end cap, a pin positioned within the housing between the power source and the end wall, a circuit wire positioned within the housing between the distal end and the end cap, and a plate attachment positioned on the end wall between the first extension walls. According to an aspect of the present disclosure the circuit wire may be in electrical contact with the contact plate and an end of the power source received by the end cap. According to another aspect of the present disclosure, the pin and the contact plate are configured to be electrically connected outside of the housing and close an electrical circuit of the disposable handle assembly such that power is supplied by the power source through the electrical circuit.

According to an aspect of the present disclosure, a testing device includes a senor having a first contact and a second contact, and a sensor housing. The sensor housing may include a first body that defines a first groove extending in a first direction, a second body extending from first body in a second direction perpendicular to the first direction, sidewalls extending from the first body and the second body, and resilient support legs extending from the first body at an angle relative to an axis of the second direction. According to an aspect of the present disclosure, the sensor may be positioned between the side walls and the first contact and the second contact extend through the second body, and the first groove is configured to receive and be attached to a rod of an assembly such that the testing device is configured to be mounted on to the assembly.

According to another aspect of the present disclosure, an assembly includes a disposable handle assembly including a power source, an electrical circuit, and a rod attached to a distal end of the disposable handle assembly, and a testing device including a sensor, a first contact and a second contact attached to the sensor, and a sensor housing mounted on to the rod. According to an aspect of the present disclosure, the first contact and the second contact may be configured to selectively close the electrical circuit such that power is supplied from the power source through the electrical circuit and to the sensor, and the sensor may be configured to emit at least one of a light signal, an audible signal, and a vibratory signal, or cause an electro-chemical reaction in response to the electrical circuit being closed.

According to still another aspect of the present disclosure, a method of preparing a disposable handle assembly for a medical procedure includes gripping the disposable handle assembly such that at least a first finger of a user is positioned on a front side of the disposable handle assembly including grooves. The method may further include rotating a testing device that is attached to a rod at a distal end of the disposable handle assembly about the rod towards the front side with a second finger until the testing device rests on an edge of an end wall of the disposable handle; and applying a downward force to the testing device with the first finger until the testing device detaches from the rod.

Certain aspects of a disposable handle assembly and a testing device have been outlined such that the detailed description thereof herein may be better understood. There are, of course, additional aspects of the disclosure that will be described below. In this respect, before explaining at least one aspect of the disposable handle assembly and the testing device in detail, it is to be understood that the disposable handle assembly and testing device are not limited in application to the details of construction and to the arrangements of the components set forth in the following description or illustrated in the drawings. Each of the disposable handle assembly and testing device are capable of functions in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the disclosure. It is important, therefore, that the claims be regarded as including such equivalent constructions insofar as they do not depart from the spirit and scope of the disclosure.

### BRIEF DESCRIPTION OF THE DRAWINGS

FIG. 1A illustrates a front perspective view of a disposable handle assembly and a testing device, according to an aspect of the present disclosure.

FIG. 1B illustrates a rear perspective view of a disposable handle assembly and a testing device, according to an aspect of the present disclosure.

FIG. 2 illustrates an exploded view of a disposable handle assembly and a testing device, according to an aspect of the present disclosure.

FIG. 3 illustrates an elevation view of a housing of the disposable handle assembly of FIG. 2, taken along section line 3-3.

FIG. 4 illustrates a cross-sectional view taken along section line 4-4 shown in FIG. 3, as applied to the disposable handle assembly and testing device of FIG. 2 in an assembled state.

FIG. 5A illustrates an enlarged portion of FIG. 4, identified as FIG. 5A, which shows a testing device in a normal state.

FIG. 5B illustrates a testing device in an actuated stated, according to an aspect of the present disclosure.

FIG. 6A illustrates a perspective view of a testing device, according to an aspect of the present disclosure.

FIG. 6B illustrates a front elevation view of a testing device, according to an aspect of the present disclosure.

FIG. 7 illustrates a cross-sectional view of the testing device of FIG. 6B, taken along section line 7-7.

FIG. 8 illustrates a side elevation view of a testing device prior to assembly, according to an aspect of the present disclosure.

FIG. 9A illustrates an elevation view of a disposable handle assembly and a testing device in a packaging, according to an aspect of the present disclosure.

FIG. 9B illustrates an elevation view of a disposable handle assembly in a packaging with a testing device being actuated, according to an aspect of the present disclosure.

FIG. 10A illustrates a front perspective view of a disposable handle assembly with a testing device attached to the disposable handle assembly, according to an aspect of the present disclosure.

FIG. 10B illustrates a front perspective view of a disposable handle assembly with a testing device detached from the disposable handle assembly, according to an aspect of the present disclosure.

FIG. 10C illustrates a front perspective view of a disposable handle assembly with a medical instrument attached to the disposable handle assembly, according to an aspect of the present disclosure.

### DETAILED DESCRIPTION

It is noted that as used herein the specification and the appending claims the singular forms “a,” “an,” and “the” can include plural references unless the context clearly dictates otherwise. Further, unless specified otherwise, the terms “substantial” or “substantially” as used herein mean “considerable in extent,” or “largely but not necessarily wholly that which is specified.” Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

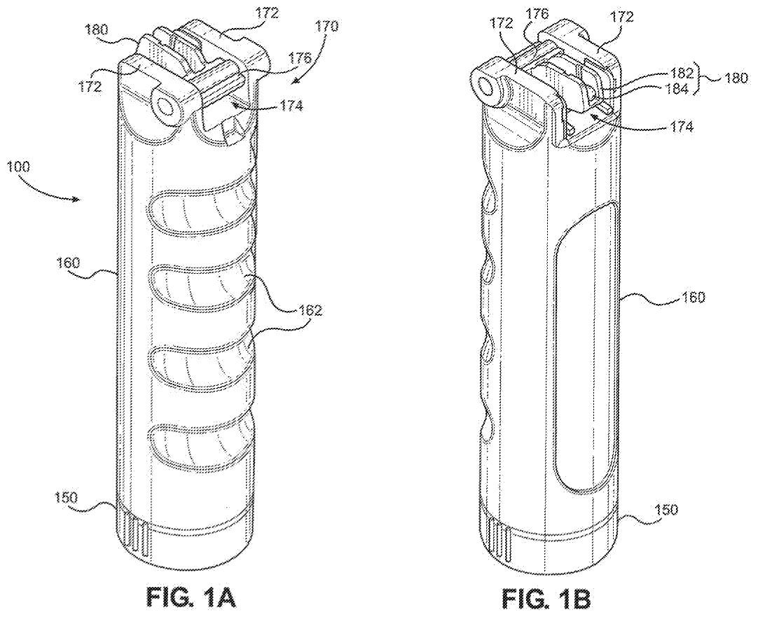

FIGS. 1A and 1B respectively illustrate front and rear perspective views of a disposable handle assembly 100 and a testing device 180, according to an aspect of the present disclosure. The disposable handle assembly 100 includes an end cap 150 that is attached to a housing 160. As described in more detail below, the end cap 150 may be attached to the housing 160 one time, and upon removal cannot be reattached to ensure the disposable handle assembly 100 is used as a single-use medical device handle. The housing 160 includes outer grooves 162 shaped to corresponding to fingers of a user so that a user may better grip the disposable handle assembly 100 during use in a medical procedure. It will be understood that the housing 160 may be provided with a flat surface instead of having the outer grooves 162.

The housing 160 includes a housing distal end 170, and the housing distal end 170 includes first housing extension walls 172. The first housing extension walls 172 define a channel 174 there between. A rod 176 extends between, and is connected to each of, the first housing extension walls 172. The testing device 180 is attached to the rod 176 as illustrated in FIGS. 1A and 1B. The testing device 180 is an assembly that includes a sensor housing 182 and a sensor 184 that is detachably or permanently mounted to the sensor housing 182. The sensor 184 may include an LED bulb that emits light when power is supplied thereto. According to an aspect of the present disclosure, the sensor may be any type of component that emits a signal that is visible, audible, and/or vibratory such that the signal may be appreciated by a person and/or user of the disposable handle assembly 100. Alternatively, according to an aspect of the present disclosure, actuation of the testing device 180 may cause an electro-chemical reaction to occur. As described in more detail below, the testing device 180 may rotate about the rod 176 relative to the housing distal end 170.

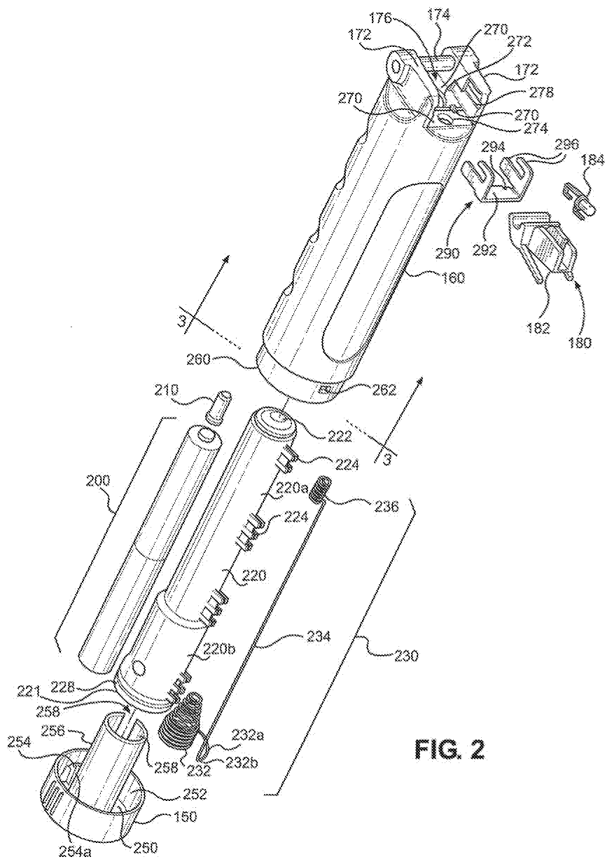

FIG. 2 illustrates an exploded view of the disposable handle assembly 100 and testing device 180, according to an aspect of the present disclosure. As illustrated in FIG. 2, the disposable handle assembly 100 includes a power source 200 and a pin 210. The power source 200 may include one or more batteries having terminals at opposite ends. In particular, the power source 200 may include two AA batteries according to an aspect of the present disclosure. However, one ordinary skill in the art will recognize that different numbers, sizes, and types of batteries (e.g. zinc-carbon, alkaline, lithium ion), or different type of power sources (e.g. terminals that receive an electrical connector) may be provided. In applications of the disposable handle assembly 100 including medical instruments such as, for example laryngoscopes, the power source 200 and the remainder of the disposable handle assembly 100, as well any laryngoscope attached, are ISO 7376 compatible.

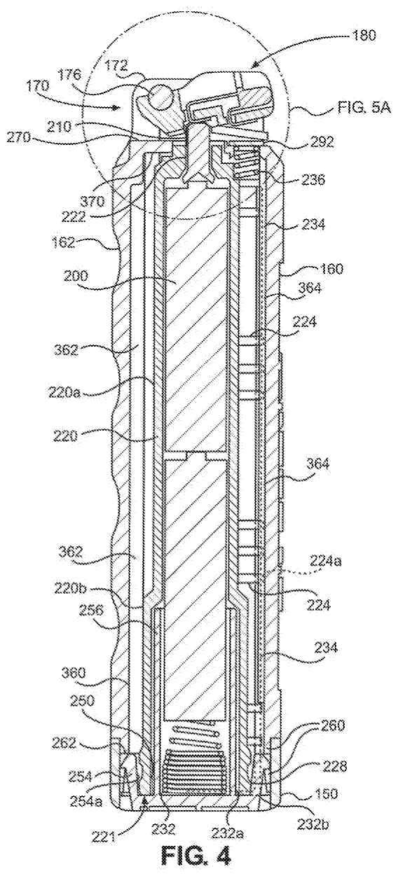

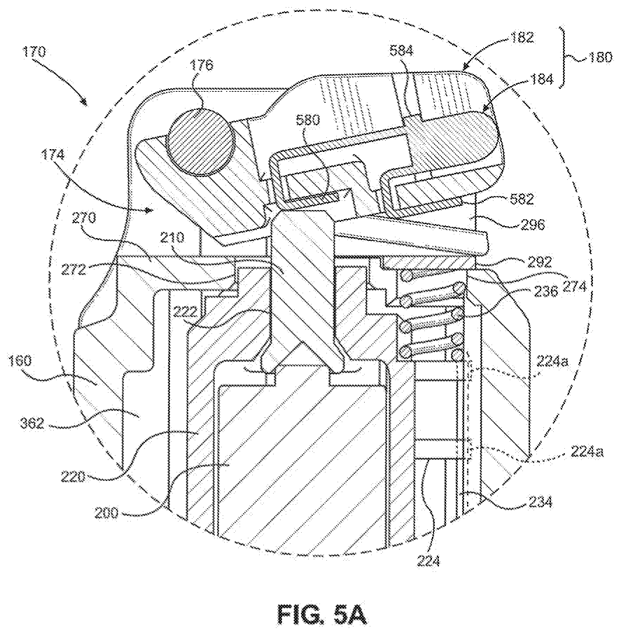

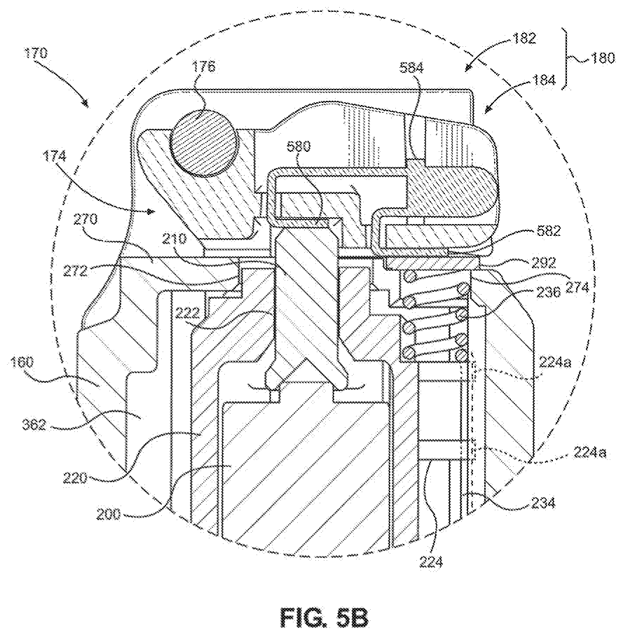

The power source 200 and the pin 210 may be positioned within an insert 220. According to an aspect of the present disclosure, the pin 210 may be mounted on the power source 200 and extend through an insert aperture 222. In particular, an insert base 221 of the insert 220 defines an open end that receives the pin 210 and the power source 200 so as to be housed within the insert 220 with the pin 210 extending through an insert aperture 222. As described in more detail below with reference to FIGS. 4 and 5, the pin 210 may be in direct contact with a terminal of the power source 200 as part of an electrical circuit, and extend through the insert aperture 222.

An exterior of the insert 220 may include a plurality of rail extensions 224 extending from an exterior cylindrical surface of the insert 220, and an insert protruding edge 228 surrounding the insert base 221. Each rail extension 224 may include two rows of prongs, the prongs of each row being spaced along a longitudinal axis of the insert 220, and a slot being defined between the two rows. The insert protruding edge 228 may be formed from a same material as the remainder of the insert 220, or a different material that is flexible (e.g. an elastomeric material) as compared to the remainder of the insert 220. In addition, the insert protruding edge 228 may have a generally semi-circular cross-section that is raised from a main exterior surface of the insert 220. Alternatively, the insert base 221 may include two or more raised circumferential edges defining grooves there between, and the insert protruding edge 228 may be provided by a ring (e.g an O-ring) formed from an elastomeric material and positioned within the grooves. As described in more detail below with reference to FIGS. 4 and 5, the rail extensions 224 may receive and hold in place a wire 234, and the insert protruding edge 228 may engage first protrusions 250 extending from the end cap 150.

The wire 234 that may be held in place by the rail extensions 224 constitutes a straight section of a circuit wire 230 that also includes a first spring 232 and a second spring 236. The wire 234 may extend between, and formed as one-piece with, the first spring 232 and the second spring 236. The first spring 232 is sized to fit within insert 220, along with the power source 200 and the pin 210. In particular, a body of the insert 220 may include a first section 220a having dimensions (e.g. length, diameter, etc.) that correspond to dimensions of the power source 200, and a second section 220b having dimensions (e.g. length, diameter, etc.) that correspond to dimensions, for example a length and outermost diameter, of the first spring 232. According to an aspect of the present disclosure, the power source 200, pin 210, insert 220, and circuit wire 230 may be assembled into a sub-component of the disposable handle assembly 100 that is inserted into the housing 160.

In order to assemble the disposable handle assembly 100, the power source 200, pin 210, insert 220, and circuit wire 230 are inserted into the housing 160 as a sub-component which may or may not include the end cap 150. The end cap 150 includes a cap end wall 250 from which a cap inner wall 252 extends along with first protrusions 254. The cap inner wall 252 may be a cylindrical wall that surrounds the first protrusions 254 and a second protrusion 256 that is positioned radially inward of inner surfaces 254a of the first protrusions 254. The second protrusion 256 may be cylindrical in shape and define a slot 258 configured to allow a bottom connecting portion 232a of the first spring 232 to pass through the slot 258. The bottom connecting portion 232a extends from a corner 232b of the circuit wire 230 between the first spring 232 and the wire 234.

In an assembled state of the disposable handle assembly 100, an annular space between the inner surfaces 254a and the second protrusion 256 is configured to receive the insert base 221, and the inner surfaces 254a of the first protrusions 254 are configured to engage the insert protruding edge 228 of the insert 220. In addition, the second protrusion 256 is configured to be positioned radially between the power source 200 and a wall of the insert 220 (FIG. 4). According to an aspect of the present disclosure, the power source 200, the pin 210, the insert 220, the circuit wire 230, and the end cap 150 may be assembled into a sub-component and positioned within the housing 160. According to another aspect of the present disclosure, the power source 200, the pin 210, the insert 220, and the circuit wire 230 may be assembled into a sub-component and positioned within the housing 160, and the end cap 150 may then be installed onto the housing 160. In the latter assembly scheme, the slot 258 is aligned with an end of the bottom connecting portion 232a immediately extending from the first spring 232, and the second protrusion 256 is positioned concentrically between the power source 200 and the insert 220.

In either assembly scheme discussed above, the end cap 150 is attached to the housing 160 to complete an assembly of the disposable handle assembly 100. In particular, the end cap 150 is positioned such that the first protrusions 254 and the second protrusion 256 are inserted into a second housing extension wall 260 on a proximal end of the housing 160, and the first protrusions 254 are received by first housing apertures 262 formed within the second housing extension wall 260. The first protrusions 254 are configured to elastically move radially inward and outward as the end cap 150 is attached to the housing 160. According to an aspect of the present disclosure, ends the first protrusions 254 may contact an inner surface of the second housing extension wall 260 as the end cap 150 is moved towards the housing distal end 170. Contact with the inner surface causes the first protrusions 254 move radially (bend or flex) inward until the ends of the first protrusions 254 engage the first housing aperture 262 and move radially outward in a spring-like movement (e.g. snap into the first housing apertures 262).

The end cap 150 is configured to be fixedly attached to the housing 160 a single time, and can only be removed from the housing 160 with a rotation of the end cap 150 which causes the first protrusions 254 to break away from the cap end wall 250. According to an aspect of the present disclosure, the end cap 150 and housing 160 are configured in accordance with a plastic end cap including at least one plastic frangible member and a hand grip of a handle as disclosed in International Patent Application No. PCT/IL2014/050745 and corresponding U.S. patent application Ser. No. 14/4173,498, the entire disclosures of which are herein incorporated by reference in their entirety. Removing the end cap 150 allows for the power source 200 to be recovered and used in another application.

As illustrated in FIG. 2, the housing distal end 170 includes a housing end wall 270 that, along with the first housing extension walls 172, defines the channel 174. A second housing aperture 272 is formed in the housing end wall 270 in a location corresponding to the pin 210 once inserted into the housing 160. A third housing aperture 274 is formed in the housing end wall 270 in a location corresponding to the second spring 236 once inserted into the housing 160. In addition, fourth housing apertures 276 are formed in the housing end wall 270. According to an aspect of the present disclosure, the fourth housing aperture 276 and recessed walls 278 formed in each of the first housing extension walls 172 are configured for engagement with a plate attachment 290.

As illustrated in FIG. 2, the plate attachment 290 includes a contact plate 292, prongs 294 extending downward at substantially right angles from the contact plate 292, and legs 296 which extend from a front to a rear of the plate attachment 290. The plate attachment 290 may be removably positioned within the channel 176 with the prongs 294 being received in the fourth housing apertures 276, and the legs 296 received in the recessed walls 278. As illustrated in FIG. 2, a shape of the legs 296 may correspond to a profile of the recessed wall 278. The contact plate 292 forms part of an electrically conductive circuit, as defined below, of the disposable handle device 100 by being in contact (i.e. compressing) the second spring 236 extending through the fourth housing aperture 276. In addition, the testing device 180 may rest on the contact plate 292 once attached to the rod 174 and rotated in a direction away from the external grooves 162 of the housing 160.

As described in more detail with reference to FIGS. 4 and 5, the contact plate 292 forms part of the electrically conductive circuit (hereafter referred to as “the electrical circuit”) of the disposable handle assembly 100 that includes: the power source 200; the pin 210; the circuit wire 230; the plate attachment 290; and an electrical connection between the pin 210 and the plate attachment 290 that may be provided by various types of components such as, for example, the testing device 180 or other medical devices or instruments.

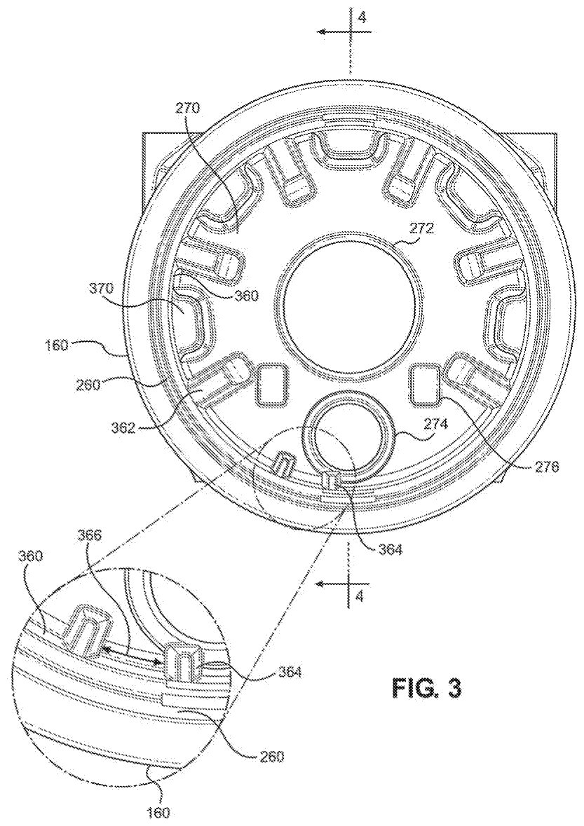

FIG. 3 illustrates an elevation view of the housing 160 of the disposable handle assembly 100 of FIG. 2, taken along section line 3-3. A longitudinal wall 360 defines an inner surface of the housing 160, and first guide rails 362 and second guide rails 364 are formed extending radially inward from the longitudinal wall 360. The first guide rails 362 and second guide rails 364 may extend longitudinally along the longitudinal wall 360 from a location corresponding to a distal edge of the second housing extension wall 260 to the housing end wall 270. As the insert 220 is positioned within the housing 160, the first guide rails 362 may guide the insert 220 via engagement with the second section 220b that centers the insert 220 within the housing 160. The second guide rails 364 may have a different shape and size relative to the first guide rails 362 so as to define a longitudinal channel 366 that receives the rail extensions 224 and the wire 234 when the insert 220 is positioned within the housing 160. In addition, stoppers 370 may be formed on the end wall 270 extending in a proximal direction, and thereby limit a movement of the insert 220 in a distal direction as the insert 220 is positioned in the housing 160.

FIG. 4 illustrates a cross-sectional view taken along section line 4-4 shown in FIG. 3, as applied to the disposable handle assembly 100 and testing device 180 of FIG. 2 in an assembled state. As illustrated in FIG. 4, the insert base 221 abuts the cap end wall 250 and the insert protruding edge 228 is friction fitted to the inner surfaces 254a of the first protrusions. The first protrusions 254 are engaged to the first housing apertures 262 formed in the second housing extension wall 260 and thereby attach the end cap 150 to the housing 160. The second housing extension wall 260 is situated between the inner cap wall 252 and the first protrusions 254. As further illustrated in FIG. 4, a wall defined by the second section 220b of the insert 220 is sandwiched between the second protrusion 256 and one of the first guide rails 362 extending from and along the longitudinal wall 360 of the housing 160.

FIG. 4 also illustrates the circuit wire 230. In particular a transition from the bottom connecting section 232a and corner 232b of the first spring 232 to the wire 234 is illustrated in FIG. 4. Although the slot 258 of the second protrusion 256 is not shown, one of ordinary skill in the art will recognize that the bottom connecting section 232a extends through the slot 258 and wraps around the second section 220b of the insert 220 to the corner 232b, from which the wire 234 extends vertically. The wire 234 extends vertically between the rail extensions 224 and the longitudinal wall 360 within the guide channel 366 (not shown) from the first spring 232 to the second spring 236. As illustrated in FIG. 4, outermost ends 224a of the rail extensions 224, and at least a portion of the wire 234 are positioned between the second guide rails 364 extending from the longitudinal wall 360.

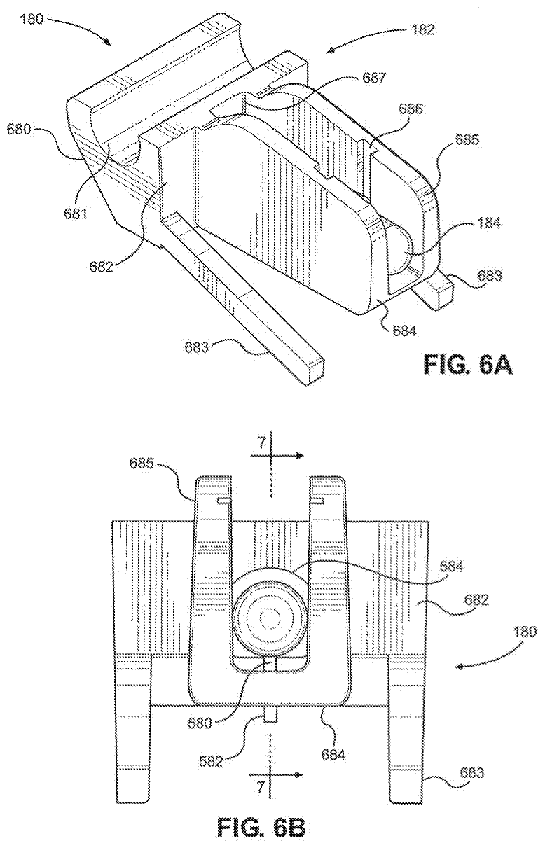

As further illustrated in FIG. 4, and described in more detail with reference to FIGS. 5A and 5B, the pin 210 extends through the insert aperture 222 and the second housing aperture 272, into the channel 174, and contacts the testing device 180 in the housing distal end 170. Resilient support legs 683 (FIGS. 6A-8) of the testing device 180 may rest on the contact plate 292 of the plate attachment 290, with the testing device 180 attached to the housing distal end 170 via an attachment to the rod 176.

FIG. 5A illustrates an enlarged portion of FIG. 4, identified as FIG. 5A. As illustrated in FIG. 5A, the wire 234 extends within the second guide rails 364, past a distal-most rail extension 224, and transitions into the second spring 236. The second spring 236 is in contact with, and may be compressed by, the contact plate 292 of the plate attachment 290. As further illustrated in FIG. 5A, the pin 210 extends through the insert aperture 222 and the second housing aperture 272 and contacts a first contact 580 of the testing device 180. A second contact 582 of the testing device 180 extends from the sensor 184 and is positioned in front of the first contact 580. In a first position of the testing device 180 illustrated in FIG. 5A, the second contact 582 is spaced from the contact plate 292 and the testing device 180 (the sensor 184) is in an normal state (i.e. un-actuated state).

FIG. 5B illustrates the testing device 180 in an actuated stated, according to an aspect of the present disclosure. More specifically, the testing device 180 has been rotated about the rod 176 downward towards the housing end wall 270 to a second position in which the second contact 582 is pressed against the contact plate 292. As illustrated in FIG. 5B, the pin 210 is depressed down against the force of the first spring 232 so as to be positioned more inside of the housing 160. In this position, a portion of the electrical circuit of the disposable handle assembly 100 between the pin 210 and the circuit wire 230 is closed through the sensor 184. The contact between the pin 210 and the first contact 580, and the contact between the second contact 582 and the contact plate 292 forms an electrical connection between the pin 210 and the contact plate 292. The electrical connection provided there between closes the electrical circuit of the disposable handle assembly 100 and causes power to be supplied to the sensor 180 from the power source via the electrical circuit, and actuate the sensor 184 of the testing device 180. Thus, the electrical circuit of the disposable handle assembly 100 is closed outside of the housing 160 with the electrical connection between the pin 210 and the contact plate 292 provided by the first contact 580 and the second contact 582.

FIG. 6A illustrates a perspective view of the testing device 180, according to an aspect of the present disclosure. As illustrated in FIG. 6A, the sensor housing 182 includes a first body 680 in which a first groove 681 is formed extending in a first direction. The first groove 681 is configured to receive the rod 176 of the disposable handle assembly 100. According to an aspect of the present disclosure, the testing device 180 may be attached to the disposable handle assembly 100 via a snap fit between the first groove 681 and the rod 176.

The first body 680 of the sensor housing 182 includes a front wall 682 from which a pair of the resilient support legs 683 and a second body 684 extend. The resilient supports legs 683 extend at an oblique angle relative to a plane of the front wall 682, whereas the second body 684 extends along a respective plane that is perpendicular to the plane of the front wall 682. The second body 684 extends in a second direction perpendicular to the first direction of the first groove 681. Thus, the resilient supports legs 683 are angled downward relative to the second body 684. The sensor 184 may be positioned between side walls 685 of the second body 684. Each side wall 685 includes a second groove 686 configured to receive the sensor protruding edge 584. A third groove 687 may be defined by the front wall 682 between the sidewalls 685 and accommodate a portion of the second contact 582.

FIG. 6B illustrates a front elevation view of the testing device 180, according to an aspect of the present disclosure. As illustrated in FIG. 6B, the resilient support legs 683 extend from an elevational position of the second body 684 downward to an elevational position below the second body 684. As result, when the testing device 180 is mounted onto the disposable handle assembly 100, the resilient support legs 683 rest on the contact plate 292. In the absence of a force pressing the second body 684 toward the housing end wall 270 and the contact plate 292, the resilient support legs 683 hold the second body 684 in a position spaced from the contact plate 292 and prevent contact between the second contact 582 and the contact plate 292.

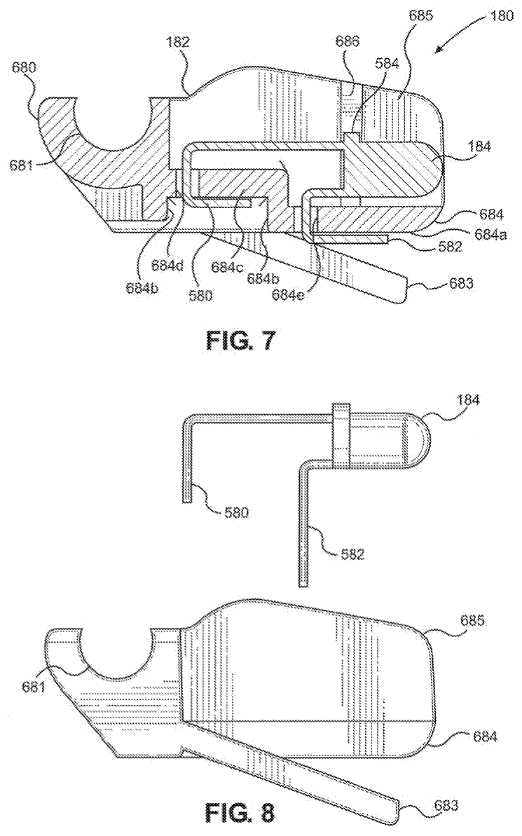

FIG. 7 illustrates a cross-sectional view of the testing device 180 of FIG. 6B, taken along section line 7-7. As illustrated in FIG. 7, the sensor protruding edge 584 is received in the second groove 686 defined by the sidewall 685. As further illustrated in FIG. 7, the second body 684 includes a bottom surface 684a, and a bottom recess 684b is formed in the bottom surface 684a as defined by a stepped wall 684c of the second body 684. The stepped wall 684c further defines a first testing device aperture 684d. The first contact 580 may be extended through and be bent around the first testing device aperture 684d such that a portion of the first contact 580 extends parallel to the stepped wall 684c. A second testing device aperture 684e is defined in the bottom surface 684a. As illustrated in FIG. 7, the second contact 582 may extend through, and be bent around the second testing device aperture 684e such that a portion of the second contact 582 extends parallel to the bottom surface 684a.

FIG. 8 illustrates a side elevation view of the testing device 180 prior to assembly, according to an aspect of the present disclosure. During assembly, the first contact 580 and the second contact 582 of the sensor 184 may be passed through the first testing device aperture 684d and the second testing device aperture 684e, respectively. The first contact 580 may be formed of a ductile metal material and bent around the first testing device aperture 684d such that a portion of the first contact 580 extended past the first testing device aperture 684d is parallel with the stepped wall 684b. Similarly, the second contact 582 may be formed of a ductile metal material and bent around the second testing device aperture 684e such that a portion of the second contact 582 extended past the second testing device aperture 684e is parallel with the bottom surface 684a of the second body 684.

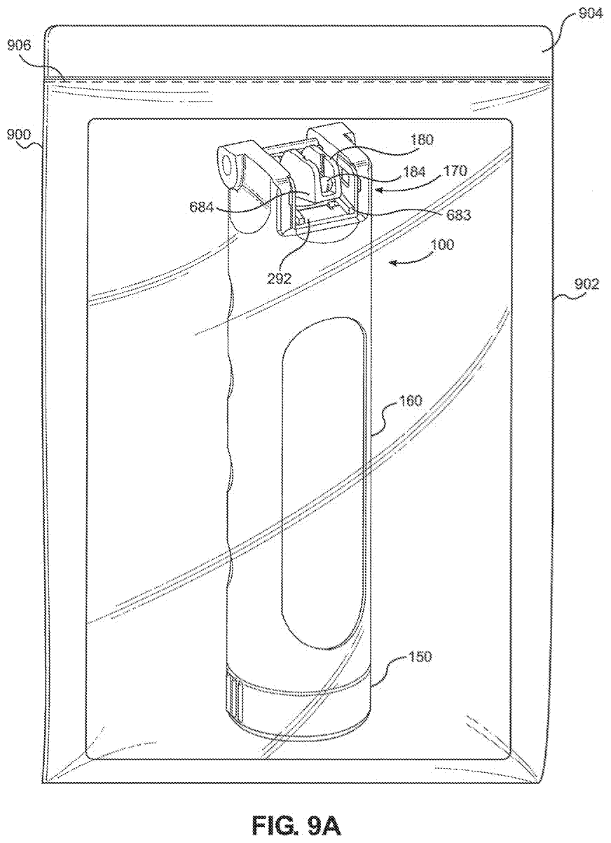

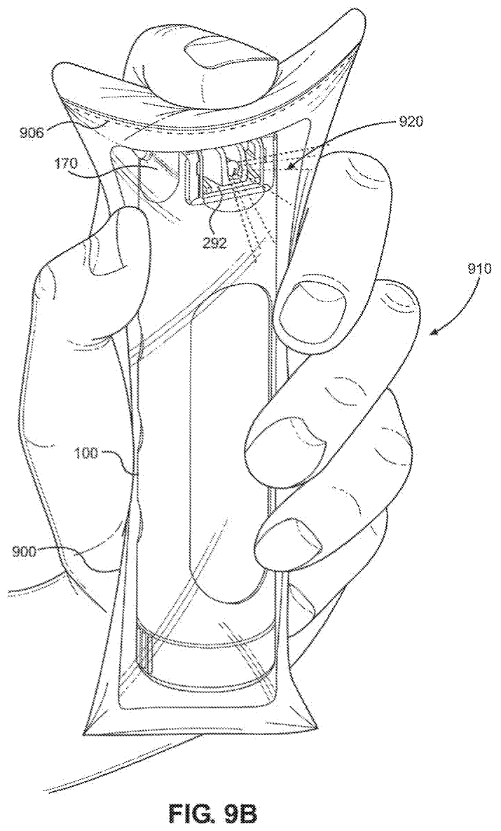

FIG. 9A illustrates a perspective view of the disposable handle assembly 100 and the testing device 180 in a packaging 900, according to an aspect of the present disclosure. The packaging 900 may be made from a transparent material such that a signal from the testing device 180 is visible through a packaging wall. According to aspect of the present disclosure, the entire packaging 900 may be transparent, or only a region of the packaging 900 corresponding to a general location of the testing 180 may be transparent, or the packaging 900 may include graphics or designs. In the case of the sensor 184 that is an LED, the packaging 900 may be of a color or a design that allows for a signal from the testing device 180 being actuated, to be be visible to a user holding, or a person with a view of the packaging 900, from an appreciable distance.

The packaging 900 may include a main body 902 and a strip 904, with a perforated section 906 between the main body 902 and the strip 904. Subsequent to an actual placement of the disposable handle assembly 100 in the packaging 900 having an open end, the packaging may be sealed and perforated and thereby defining the main body 902 and the strip 904. One of ordinary skill in the art will recognize that the housing distal end 170 may be on a side of the packaging 900 including the strip 904, or the end cap 150 may be on a side with the strip 904.

According to an aspect of the present disclosure, two perforated sections 906 and two strips 904 may be provided on opposite sides of the packaging, or the perforated section 906 and strip 904 can extend along a long edge of the packaging (i.e. extending up and down, parallel to a longitudinal axis of the disposable handle assembly 100). One of ordinary skill in the art will recognize that the perforated section 906 may be provided in the middle of the packaging. In any configuration, the perforated section 906 of the present disclosure is provided in manner that maintains the interior of the packaging 900 as being closed from an exterior of the packaging 900. Accordingly, the interior of the packaging 900 is, and remains, a closed environment that the disposable handle assembly 100 is placed within. Thus, elements (fluid, solid particles, etc) are prevented from entering into the interior of the packaging 900 and coming into contact with the disposable handle assembly 100. As a result, the disposable handle assembly 100 is maintained in a state suitable (i.e. safe) for an intended use for a particular procedure. For example, the disposable handle assembly 100 is maintained within the packaging 900 in state such that the disposable handle assembly 100 is safe and meets particular/regulatory requirements for use in a medical procedure.

A user may grasp the packaging 900 and pull on the strip 904 with a minimal force so that the strip 904 is easily separated from the main body 902 at the perforated section 906. Accordingly, once the packaging has been opened, the disposable handle assembly 100 may be removed for use in a medical procedure. However, prior to removing the strip 904 and opening the packaging, a user may wish to determine whether the disposable handle assembly 100 is operational via a simple procedure illustrated in FIG. 9B.

FIG. 9B illustrates a perspective view of the disposable handle assembly 100 in the packaging 900 with the testing device 180 being actuated by a hand 910 of a user, according to an aspect of the present disclosure. As illustrated in FIG. 9B the packaging 900 is grasped by the hand 910 in such a way that an index finger points in a same direction as the sensor 184 of the testing device 180. From this position, a user may use their index finger to depress the testing device 180, closing the electrical circuit of the disposable handle assembly 100 as illustrated in FIG. 5B. As a result, power from the power source 200 may be supplied to the sensor 184. More specifically, the first contact 580 is in contact with the pin 210 that is in contact with the power source 200, and the second contact 582 is in contact with the contact plate 292 that is in contact with the second spring 236.

Assuming the disposable handle assembly 100 is able to function properly, e.g. there is no issue of improper or insufficient contact between power source 200 and the pin 210 and/or the circuit wire 230, the power source 200 has not been completely used up/depleted, the power source 200 has not degraded in some manner, etc., the sensor 184 will be supplied with power and emit a signal 920 that a person from outside of the packaging 900 can observe. The testing device 180 can be actuated multiple times as long the disposable handle assembly 100 is capable of operating properly as discussed above.

According to an aspect of the present disclosure, in one example of the testing device 180, the sensor 184 is an LED or similar type of light that emits light when power is supplied thereto. FIG. 9B illustrates an example of the sensor 184 emitting the signal 920 in the form of light signal that can be observed through the packaging 900 to indicate that the disposable handle assembly 100 is capable of functioning properly during a procedure to be performed. As a result, during an ongoing procedure, a clinician can quickly test several disposable handle assembles 100 to find one that will work. Thus the clinician can select properly functioning equipment during a procedure without having to take time to open the packaging 900, remove the disposable handle assembly 100 from the packaging 900, attach a medical instrument to the disposable handle assembly 100, and actuate the medical instrument only to find out that the disposable handle assembly 100 is not operational. The clinician can test one or, if needed, more disposable handle assemblies 100 in a short period of time and quickly resume a procedure that is in process.

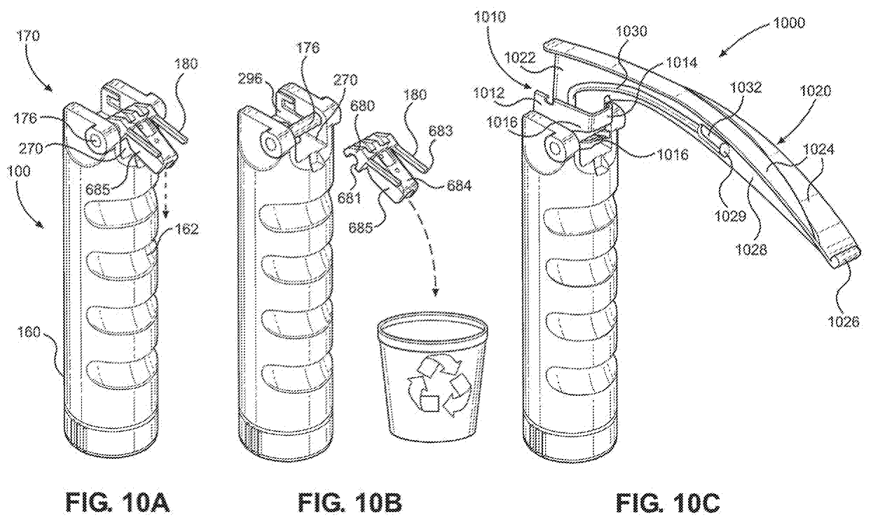

FIG. 10A illustrates a front perspective view of the disposable handle assembly 100 with the testing device 180 attached to the disposable handle assembly 100, according to an aspect of the present disclosure. As illustrated in FIG. 10A, the testing device 180 is attached to the rod 176. As part of a removal process, the testing device 180 may be rotated about the rod 176 towards the front side of the disposable handle assembly 100 which includes the grooves 162. A user may grasp the disposable handle assembly 100 as it would be grasped for a procedure, and rotate the testing device 180 via a finger (e.g. a thumb, index finger, etc.) about the rod 176. The testing device 180 may rotate such that the side walls 685 rest on an edge of the housing end wall 270 as illustrated in FIG. 10A.

FIG. 10B illustrates a front perspective view of the disposable handle assembly 100 with the testing device 180 detached from the disposable handle assembly 100, according to an aspect of the present disclosure. The testing device 180, after having been used to test the disposable handle assembly 100, within or outside of the packaging 900, may be disposed of as illustrated. Thus, the testing device 180 may not have a function to perform in a procedure for which the disposable handle assembly 100 is going to be used.

According to an aspect of the present disclosure, a user may remove the testing device 180 having the same grip as was used to rotate the testing device 180 around the rod 176. In particular, with the testing device 180 resting on an edge of the housing end wall 270 as illustrated in FIG. 10A, a user may use a finger wrapped around the housing 160 resting in one of the grooves 160 (e.g. an index finger), and apply a downward force to the second body 684 and the resilient support legs 685.

According to an aspect of the present disclosure, the user may apply the force without having to re-orientate the disposable handle assembly 100 in the user's hand. For example, in gripping the grooves 160 with the user's fingers except the thumb, the user may be able to reach the testing device 180 in the first position illustrated in FIG. 5A with the user's thumb, and rotate the testing device 180 with a flicking motion of the thumb. Without changing a grip, and with the testing device 180 resting on an edge of the housing end wall 270 as illustrated in FIG. 10A, the user may move an index finger from a respective groove 160 and easily apply a downward force on the testing device 180. A substantial downward force will not be required for the first groove 681 to become detached (e.g. snapped off of) from the rod 176 and thereby detach testing device 180 from the disposable handle 180.

FIG. 10C illustrates a front perspective view of the disposable handle assembly 100 with a medical instrument 1000 attached to the disposable handle assembly 100, according to an aspect of the present disclosure. According to an aspect of the present disclosure, once the testing device 180 is removed from the disposable handle assembly 100, the medical instrument 1000 may be attached to the housing distal end 170. In particular, the medical instrument 1000 may be an laryngoscope blade which can be used for an intubation procedure. According to one aspect of the present disclosure the medical instrument 1000 may be laryngoscope blade as described in U.S. Pat. Nos. 8,142,353 or 5,879,304, the entire disclosures of which are herein incorporated by reference in their entirety. However, one of ordinary skill in the art will recognize that the disposable handle assembly 100 may be used for attachment to other laryngoscope blades or other types of medical instruments. Further, it will be understood that the testing device 180 may be attached and used to test other types of handle assemblies or medical instruments which are kept within closed packaging and include a power source.

As illustrated in FIG. 10C, the medical instrument 1000 may include a fitting 1010 for attaching to the housing distal end 170, and a blade 1020 used, for example, in an intubation procedures. The fitting 1010 may include fitting side walls 1012 attached by a fitting front wall 1014. The fitting side walls 1012 and the fitting front wall 1014 may define a hook 1016 configured to be mounted on (e.g. snap fit on to), and permit the medical instrument to rotate about, the rod 176 of the disposable handle assembly 100. The blade 1020 includes a proximal end 1022 that may extend from one of the fitting sidewalls 1012, and a blade section 1024 (a spatula, tongue depressor, etc.) extending from the proximal end 1022 to a distal tip 1026. The blade section 1024 includes a transverse wall 1028 that includes an opening 1029.

A conduit distal end 1032 of a conduit 1030 (“light guide 1030”) may extend through the opening 1029 from one side of the transverse wall 1028 to an opposite side thereof. A proximal end (not shown) of the light guide 1030 may include or be attached to a light source (not shown). The light source may be powered through a contact or terminal (not shown) on a bottom of the fitting 1010. The contact or terminal may be arranged to contact the pin 210 protruding through the housing end wall 270 prior to being depressed downward by the bottom of the fitting 1010, once the fitting 1010 is installed on the disposable handle assembly. The contact or terminal may extend from, or be in contact with, the fitting sidewalls 1012 that may be in planar contact with the legs 296 of the plate attachment 290. The fitting sidewall 1012 may be formed of metal or some other electrically conductive material. It will be understood that the fitting sidewalls 1012 may be formed of a non-metal material with regions corresponding to at least a portion of the legs 296 and formed of, or have embedded therein, components formed of a material capable of making electrical contact with the legs 296 and the contact or terminal of the medical instrument 1000. As the legs 296 extend from the contact plate 292, which in turn is pressed against and in electrical contact with the second spring 236 of the circuit wire 230, the fitting 1010 may be positioned to close the electrical circuit of the disposable handle assembly 100.

For example, the medical instrument 1000 may be attached to the rod 176 such that the blade section 1024 is substantially parallel to a longitudinal axis of the housing 160. Upon rotation of the medical instrument 1000 about the rod 176 and away from the housing 160, the fitting sidewalls 1012 will come into electrical contact with the legs 296 and the contact or terminal of the medical instrument will come into electrical contact with the pin 210 to close the electrical circuit of the disposable handle assembly 100. Accordingly, the light source will emit light that is guided by the light guide 1030 and the conduit distal tip 1032 and illuminates an area around the distal tip 1026 of the blade 1020.

It should also be appreciated that the systems in the figures are merely illustrative and that other implementations might be used. Additionally, it should be appreciated that the functionality disclosed herein might be implemented in software, hardware, or a combination of software and hardware. Other implementations should be apparent to those skilled in the art.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

We claim:

1. A disposable handle assembly comprising:
a housing including an end wall at a distal end of the housing and first extension walls extending from the end wall;
an end cap attached to a second extension wall on a proximal end of the housing;
a power source positioned within the housing between the distal end and the end cap;
a pin positioned within the housing between the power source and the end wall;
a circuit wire positioned within the housing between the distal end and the end cap; and
a plate attachment positioned on the end wall between the first extension walls,
wherein the circuit wire is in electrical contact with the contact plate and an end of the power source received by the end cap, and
wherein the pin and the contact plate are configured to be electrically connected outside of the housing and close an electrical circuit of the disposable handle assembly such that power is supplied by the power source through the electrical circuit.

2. The disposable handle assembly of claim 1, further comprising:
an insert positioned between the end wall and the end cap along a longitudinal axis of the housing, and between the power source and the circuit wire in a radial direction;
wherein the housing includes guide rails that extend from a longitudinal wall of the housing in the radial direction, and extend along the longitudinal wall between the end wall and the second wall extension, and
wherein the insert includes rail extensions configured to hold a wire of the circuit wire between the longitudinal wall and the rails extensions and within a longitudinal channel defined by the guide rails.

3. The disposable handle assembly of claim 1,
wherein the end wall defines a first aperture and a second aperture, and
wherein the pin is configured to extend through the first aperture and the plate attachment is positioned on the end wall and covers the second aperture.

4. The disposable handle assembly of claim 3,
wherein the circuit wire includes a first spring, a second spring, and a wire extending between the first spring and the second spring,
wherein the second spring is positioned between the power source and the end cap, and
wherein the first spring is positioned within the second aperture and contacts the plate attachment covering the second aperture.

5. The disposable handle assembly of claim 1, further comprising:
a rod extending between the first extension walls,
wherein the rod is configured to be attached to a testing device that includes a sensor attached to a first contact and a second contact, and
wherein the pin is configured to engage the first contact and the plate attachment is configured to engage the second contact such that pin is electrically connected to the plate attachment through the testing device and power is supplied to the testing device to emit a signal.

6. The disposable handle assembly of claim 1, further comprising:
a rod extending between the first extension walls,
wherein the rod is configured to be attached to a medical instrument including a blade, a fitting, and a light source, and
wherein the pin and the plate attachment are configured to engage respective portions of the fitting such that pin is electrically connected to the plate attachment through the fitting and power is supplied to the light source to emit light towards a distal tip of the blade.

7. A testing device comprising;
a senor having a first contact and a second contact; and
a sensor housing,
wherein the sensor housing includes:
a first body that defines a first groove extending in a first direction,
a second body extending from first body in a second direction perpendicular to the first direction,
sidewalls extending from the first body and the second body, and
resilient support legs extending from the first body at an angle relative to an axis of the second direction,
wherein the sensor is positioned between the side walls and the first contact and the second contact extend through the second body, and
wherein the first groove is configured to receive and be attached to a rod of an assembly such that the testing device is configured to be mounted on to the assembly.

8. The testing device of claim 7,
wherein the sensor is an LED, and
wherein an electrical connection between the first contact and the second contact causes power to be supplied to the LED such that the LED emits a light signal.

9. The testing device of claim 7,
wherein the resilient support legs are elastically flexible, and
wherein the second body is configured to move relative to the resilient support legs in response to a downward force being applied to the side walls.

10. An assembly comprising:
a disposable handle assembly including a power source, an electrical circuit, and a rod attached to a distal end of the disposable handle assembly; and
a testing device including a sensor, a first contact and a second contact attached to the sensor, and a sensor housing mounted on to the rod;
wherein the first contact and the second contact are configured to selectively close the electrical circuit such that power is supplied from the power source through the electrical circuit and to the sensor, and
wherein the sensor is configured to emit at least one of a light signal, an audible signal, and a vibratory signal in response to the electrical circuit being closed.

11. The assembly of claim 10,
further comprising a packaging including a body,
wherein the disposable handle assembly is located within the body of the packaging with the testing device attached to the rod in a first position,
wherein at least the second contact is spaced from an end wall of the disposable handle assembly such that the first contact and second contact do not close the electrical circuit in the first position, and
wherein the testing device is configured to be moved from the first position to a second position pressed against the disposable handle assembly and close the electrical circuit in response to a force directed toward the end wall being applied to the testing device from outside of the packaging.

12. The assembly of claim 10,
wherein the sensor is an LED configured to emit the light signal and at least a portion of the packaging is formed from a transparent material,
wherein the packaging is configured to move in response to the force being applied and the sensor is configured to emit the light signal upon being pressed against the end wall, and
wherein the light signal is visible from outside of the packaging.

13. A method of preparing a disposable handle assembly for a medical procedure, the method comprising:
gripping the disposable handle assembly such that at least a first finger of a user is positioned on a front side of the disposable handle assembly including grooves;
rotating a testing device that is attached to a rod at a distal end of the disposable handle assembly about the rod towards the front side with a second finger until the testing device rests on an edge of an end wall of the disposable handle; and
applying a downward force to the testing device with the first finger until the testing device detaches from the rod.

14. The method of claim 13, further comprising, prior to the gripping the disposable handle assembly, the steps including:
providing the disposable handle assembly with the testing device attached to the rod in a packaging, the packaging being sealed and having a body with at least one region formed from a transparent material;
gripping the packaging and the disposable handle assembly;
applying a downward force to the testing device from outside of the packaging with at least one of the first finger and the second finger, until a contact of the testing device is pressed against the disposable handle assembly such that an electrical circuit of the disposable handle assembly is closed; and
observing, through the at least one region, a response of a sensor of the testing device to the contact being pressed against the disposable handle device.

15. The method of claim 14, further comprising determining an operational status of the disposable handle assembly based on the response of the sensor observed through the at least one region.

16. The method of claim 13, further comprising causing a medical instrument to be operative for use in the medical procedure by attaching the medical instrument to the rod of the disposable handle assembly.
